(19) United States
(12) Patent Application Publication
(10) Pub. No.: US 2020/0405653 A1
(43) Pub. Date: Dec. 31, 2020

(54) METHYLPHENIDATE-CONTAINING TRANSDERMAL PATCH

(71) Applicant: Hisamitsu Pharmaceutical Co., Inc., Tosu-shi, Saga (JP)

(72) Inventors: Naoyuki Uchida, Tsukuba-shi, Ibaraki (JP); Kazuya Kominami, Tsukuba-shi, Ibaraki (JP); Ryo Tanaka, Tsukuba-shi, Ibaraki (JP); Takito Shima, Tsukuba-shi, Ibaraki (JP)

(73) Assignee: Hisamitsu Pharmaceutical Co., Inc., Tosu-shi, Saga (JP)

(21) Appl. No.: 16/975,827

(22) PCT Filed: Feb. 18, 2019
(86) PCT No.: PCT/JP2019/005823

§ 371 Date: Aug. 26, 2020

(30) Foreign Application Priority Data
Feb. 27, 2018 (JP) 2018-032720

(51) Int. Cl. A61K 9/70; A61K 31/4458

(52) U.S. Cl. CPC A61K 9/7061; A61K 9/7069; A61K 31/4458

(57) ABSTRACT

The present invention addresses the problem of providing a methylphenidate-containing transdermal patch in which cold flow beyond the edge of the patch containing methylphenidate is inhibited, and which has sufficient physical properties as a preparation, and excellent handleability. The present invention pertains to: a transdermal patch comprising a backing layer and an adhesive layer, wherein the adhesive layer contains methylphenidate, a rubber-based adhesive agent, and an acrylic adhesive agent, and the mixing ratio of the rubber-based adhesive agent to the acrylic adhesive agent is 9:1 to 1:9, and a production method for the transdermal patch.

### TECHNICAL FIELD

The present invention relates to a patch containing methylphenidate in an adhesive layer and a method for producing the same.

### BACKGROUND ART

Methylphenidate is a central nervous system stimulant used for the treatment of attention deficit/hyperactivity disorder (AD/HD), and its tablet (Ritalin® tablet) or controlled release-type sustained-release tablet (Concerta® tablet) are commercially available. As a dosage form other than the oral preparation, Daytrana®, which is a patch, is commercially available overseas.

Several embodiments have been studied as a patch containing methylphenidate; for example, a patch using a mixture of a silicone adhesive, an acrylic-based adhesive and methylphenidate for transdermal delivery of methylphenidate to AD/HD patients for at least 10 hours has been proposed (Patent Document 1).

In addition, regarding the above Daytrana® there are problems that when a release liner is removed from a polymer matrix containing acrylic polymer, silicone polymer and methylphenidate, the matrix is damaged (Patent Document 2), and that a release liner cannot be peeled off satisfactorily because release force from the release liner increases with time (Patent Document 3); in order to address such problems, the following has been proposed: various acrylate monomers are mixed in specific ratios (Patent Document 2), and a rubber-based polymer such as styrene-isoprene-styrene block copolymer is used (Patent Document 3).

Meanwhile, in the field of patches, a problem of cold flow due to distortion, deformation or dimensional change of the adhesive matrix under storage conditions has been generally known (Patent Document 4). Furthermore, it has been proposed to control drug release and transdermal absorbability by incorporating calcium silicate into the patch (Patent Document 5).

### CITATION LIST

### Patent Document

[Patent Document 1] JP A No. 2002-510600

[Patent Document 2] WO 2014/062494

[Patent Document 3] WO 2014/159573

[Patent Document 4] JP A No. 2016-504360

[Patent Document 5] JP A No. 4-108739

### SUMMARY OF INVENTION

### Problems to be Solved by Invention

While researching and developing higher-performance patches containing methylphenidate, the present inventors have faced previously unrecognized problems that, when methylphenidate is contained in a usual adhesive layer at a concentration necessary to maintain and exert the effect as an active ingredient, the adhesion strength and skin permeability are not always favorable, and in particular cold flow possibly occurs. Therefore, the object of the present invention is to solve these problems, and to provide a patch containing methylphenidate, which is excellent in handling and has sufficient physical properties as a preparation without occurrence of cold flow.

### Means for Solving the Problems

The present inventors have conducted extensive studies to solve such problems, and found that, in a patch containing methylphenidate, by forming an adhesive layer using a rubber-based adhesive and an acrylic-based adhesive at a predetermined mixing ratio, a patch containing methylphenidate which is excellent in handling, and which suppresses cold flow of the patch and has sufficient physical properties as a preparation can be obtained; as a result of further studies, the present inventors have completed the present invention. That is, the present invention relates to the following.

[1] A patch comprising a backing layer and an adhesive layer, wherein
the adhesive layer contains methylphenidate, a rubber-based adhesive and an acrylic-based adhesive, and
the mixing ratio of the rubber-based adhesive and the acrylic-based adhesive is 9:1 to 1:9.

[2] The patch according to [1], wherein the rubber-based adhesive contains a styrene-isoprene-styrene block copolymer.

[3] The patch according to [1] or [2] , wherein the rubber-based adhesive contains a tackifying resin.

[4] The patch according to any one of [1] to [3], wherein the acrylic-based adhesive does not have a carboxyl group.

[5] The patch according to any one of [1] to [4], wherein the rubber-based adhesive contains an alicyclic hydrocarbon resin and/or a terpene-based resin as a tackifying resin.

[6] The patch according to any one of [1] to [5], wherein the adhesive layer contains methylphenidate in a proportion of 10 to 30 mass % relative to the total amount of the adhesive layer.

[7] The patch according to any one of [1] to [6], wherein the mixing ratio of the rubber-based adhesive and the acrylic-based adhesive is 9:1 to 3:7.

[8] The patch according to any one of [5] to [7], wherein the mixing ratio of the alicyclic hydrocarbon resin and the terpene-based resin is 2:1 to 1:2.

### Advantageous Effects of Invention

According to the present invention, a patch containing methylphenidate can provide sufficient physical properties as a preparation and excellent handleability while suppressing cold flow. In particular, in an embodiment wherein the patch contains calcium silicate, phase separation between the rubber-based adhesive and the acrylic-based adhesive can be suppressed, and further excellent physical properties as a preparation and handleability can be obtained. Therefore, the patch can be stably stored and distributed, and can be stably used by AD/HD patients.

### Embodiments for Carrying out Invention

The patch of the present invention comprises, for example, a backing layer and an adhesive layer laminated on the backing layer.

The backing may be any one that can maintain the shape of the patch, especially the adhesive layer. Examples of a material of the backing include polyethylene, polypropylene, polybutadiene, ethylene-vinyl chloride copolymer, polyvinyl chloride, polyamide such as Nylon (trade name), polyester, cellulose derivative, synthetic resin such as polyurethane, etc. Nature of the backing is, for example, a film, a sheet, a sheet-like porous body, a sheet-like foam, a textile such as a woven fabric, a knitted fabric and a non-woven fabric, etc., and a laminate thereof. The thickness of the backing is not particularly limited, and usually it is preferably about 2 to 3000 μm.

The adhesive layer contains methylphenidate, a rubber-based adhesive and an acrylic-based adhesive, and the mixing ratio of the rubber-based adhesive and the acrylic-based adhesive is preferably 9:1 to 1:9. Furthermore, the patch of the present invention may contain, in addition to methylphenidate, the rubber-based adhesive base and the tackifying resin, if necessary, a plasticizer, an absorption promoter, a stabilizer, a solubilizer, a crosslinking agent, a preservative, a filler, and other additive components such as fragrances.

The methylphenidate of the present invention may be any isomers of methylphenidate including stereoisomers (d-erythro-methylphenidate, 1-erythro-methylphenidate, d-threo-methylphenidate, and 1-threo-methylphenidate), or a derivative or salt thereof; in addition, it is interchangeable with methylphenyl(piperidin-2-yl)acetate, and may be a derivative or salt thereof. The methylphenidate of the present invention may also be a mixture of two or more racemic compounds (such as d/1-erythro-methylphenidate and d/1-threo-methylphenidate).

The content of the above methylphenidate can be appropriately set by those skilled in the art, and based on the total amount of the adhesive layer, it is preferably 10 to 30 mass %, more preferably 15 to 30 mass %, furthermore preferably 18 to 27 mass %, and particularly preferably 20 to 25 mass %.

The rubber-based adhesive of the present invention contains a rubber-based adhesive base and may further contain a tackifying resin.

The rubber-based adhesive base of the present invention may contain styrene-isoprene-styrene block copolymer (hereinafter abbreviated as “SIS”), natural rubber, synthetic rubber, isoprene rubber, polyisobutylene, styrene-butadiene-styrene block copolymer, styrene-butadiene rubber, polybutene, alkyl vinyl ether (co) polymer, polyisoprene, polybutadiene, styrene-butadiene copolymer, styrene-isoprene copolymer and the like. The rubber-based adhesive base of the present invention may be used alone or in combination of two or more.

Specific examples of the rubber-based adhesive base include Oppanol B12, B15, B50, B80, B100, B120, B150, B220 (by BASF, trade name), JSR butyl 065, 268, 365 (by JSR Corporation, trade name), Vistanex LM-MS, MH, H, MML-80, 100, 120, 140 (by Exxon Chemical Company, trade name), HYCAR (by Goodrich Chemical Co., trade name), SIBSTAR T102 (by Kaneka Corporation, trade name) and the like.

In the rubber-based adhesive of the present invention, a rubber-based adhesive base may be used alone or two or more kinds of rubber-based adhesive bases may be used in combination, and they may be used by further combining one or two or more tackifying resins. In the rubber-based adhesive of the present invention, it is preferable to combine SIS and a terpene-based resin and/or an alicyclic saturated hydrocarbon resin, and particularly preferable to combine SIS, an alicyclic saturated hydrocarbon resin and a terpene-based resin. The content of the rubber-based adhesive can be appropriately set by those skilled in the art in consideration of the sufficient adhesion strength and local irritation at the time of peeling of the patch, and it is preferably 9 to 81 mass %, more preferably 9 to 72 mass % based on the total amount of the adhesive layer.

Examples of tackifying resins that can be used in the rubber-based adhesive of the present invention include terpene-based resins, alicyclic saturated hydrocarbon resins, rosin-based resins, phenol-based resins, xylene-based resins and the like.

Specific examples of the terpene-based resin include YS resin (by Yasuhara Chemical Co., Ltd., trade name) and Picolite (by Ruth & Dilworth, trade name) and the like. Specific examples of the alicyclic saturated hydrocarbon resin include Alcon® (by Arakawa Chemical Industries, Ltd., trade name), Regalrez (by Eastman Chemical Company, trade name), Piccolastic (by Eastman Chemical Company, trade name), Escoletz (by Exxon Chemical Company, trade name), Wing Tack (by Goodyear Co., trade name), Quintone® (by Zeon Corporation, trade name) and the like. Specific examples of the rosin-based resins, phenol-based resins, and xylene-based resins include ESTER GUM (by Arakawa Chemical Industries, Ltd., trade name), Hariester (by Harima Chemicals Group, Inc., trade name), Pentalyn® (by Eastman Chemical Company, trade name), Foral (by Eastman Chemical Company, trade name), KE-311 (by Arakawa Chemical Industries, Ltd., trade name) and the like.

The above-mentioned terpene-based res ins may be used alone or in combination of two or more. In addition, the content of the terpene-based resin can be appropriately set by those skilled in the art in consideration of the sufficient adhesion strength and local irritation at the time of peeling of the patch, and it is preferably 3 to 40 mass % based on the total amount of the adhesive layer.

The above-mentioned alicyclic saturated hydrocarbon resins may be used alone or in combination of two or more. In addition, the content of the alicyclic saturated hydrocarbon resin can be appropriately set by those skilled in the art in consideration of sufficient adhesion strength and local irritation at the time of peeling of the patch, and it is preferably 3 to 40 mass % based on the total amount of the adhesive layer.

The acrylic-based adhesive of the present invention is a component that imparts adhesiveness to the adhesive layer, and is, for example, a (co)polymer of one or two or more (meth)acrylic acid alkyl esters. Examples of alkyl (meth) acrylic acid alkyl esters include butyl (meth) acrylate, isobutyl (meth)acrylate, hexyl (meth)acrylate, octyl (meth)acrylate, 2-ethylhexyl (meth)acrylate, decyl (meth)acrylate, etc. In the present specification, the term “(meth)acrylic acid” means either one or both of acrylic acid and methacrylic acid, and similar expressions are defined similarly.

The acrylic-based adhesive may be a copolymer formed from a (meth) acrylic acid alkyl ester (main monomer) and a comonomer. Examples of the main monomer include methyl (meth)acrylate, ethyl (meth)acrylate, butyl (meth)acrylate, hexyl (meth)acrylate, heptyl (meth)acrylate, octyl (meth)acrylate, 2-ethylhexyl (meth) acrylate, etc., and one of these may be used alone or two or more thereof may be used in combination. The comonomer may be any component that can be copolymerized with a (meth)acrylic acid alkyl ester. Examples of the comonomer include (meth)acrylic acid hydroxyalkyl ester, ethylene, propylene, styrene, vinyl acetate, N-vinylpyrrolidone, (meth)acrylic acid, (meth)acrylic acid amide, etc. The comonomer may be a single kind or a combination of two or more kinds.

Specific examples of the acrylic-based adhesive include acrylic acid/acrylic acid octyl ester copolymer, 2-ethylhexyl acrylate/vinylpyrrolidone copolymer solution, acrylic acid ester/vinyl acetate copolymer, 2-ethylhexyl acrylate/2-ethylhexyl methacrylate/dodecyl methacrylate copolymer, methyl acrylate/2-ethylhexyl acrylate copolymer resin emulsion, and an acrylic-based polymer contained in an acrylic resin alkanolamine solution, etc. Specific examples of such an acrylic-based adhesive include DURO-TAK series (by Henkel) such as DURO-TAK® 387-2510, DURO-TAK® 87-2510, DURO-TAK® 387-2287, and DURO-TAK® 87-2287, DURO-TAK® 87-4287, DURO-TAK® 387-2516, DURO-TAK® 87-2516, DURO-TAK® 87-2074, DURO-TAK® 87-900A, DURO-TAK® 87-901A, DURO-TAK® 87-9301, DURO-TAK® 87-4098, etc.; GELVA series (by Henkel) such as GELVA® GMS 788, GELVA® GMS 3083, GELVA® GMS 325, etc.; MAS series (by CosMED Pharmaceutical Co., Ltd.) such as MAS 811 (trade name), MAS683 (trade name), etc.; Eudragit® series (by Evonik Industries AG), Nicasol® (by Nippon Carbide Industries Co., Inc.), Ultrasol® (by Aica Kogyo Co., Ltd.).

The above acrylic-based adhesives may be used together with a rubber-based adhesive, and may be used alone or in combination of two or more. In addition, the content of the acrylic-based adhesive can be appropriately set by those skilled in the art in consideration of the sufficient adhesion strength and local irritation at the time of peeling of the patch, and based on the total amount of the adhesive layer, it is preferably 9 to 81 mass %. In addition, preferably the acrylic-based adhesive does not have a carboxyl group in the molecule. Furthermore, the molecule may not have a polar functional group and may have a hydroxy group. It is preferable to have no polar functional group and/or have a hydroxy group in the molecule.

The plasticizer may be any of those that impart flexibility to the adhesive layer. Examples of the plasticizer include mineral oils (for example, paraffin oil, naphthenic oil, aromatic oil), animal oils (for example, squalane, squalene), vegetable oils (for example, olive oil, camellia oil, castor oil, tall oil, peanut oil), silicone oils, dibasic acid esters (for example, dibutyl phthalate, dioctyl phthalate), liquid rubbers (for example, liquid polybutene, liquid polyisoprene), liquid fatty acid esters (for example, isopropyl myristate, hexyl laurate, diethyl sebacate, diisopropyl sebacate), polyhydric alcohols (for example, diethylene glycol, polyethylene glycol, propylene glycol, dipropylene glycol), triacetin, triethyl citrate, crotamiton and the like. The plasticizers may be used alone or in combination of two or more.

The above plasticizers may be used alone or in combination of two or more. In addition, the content of the plasticizer can be appropriately set by those skilled in the art in consideration of sufficient plasticity of the patch, and based on the total amount of the adhesive layer, it is preferably 0 to 15 mass %, more preferably 0.5 to 3 mass %, and particularly preferably 1 to 2 mass %.

The absorption promoter is a component that regulates the skin permeability of methylphenidate or a pharmaceutically acceptable salt thereof. The absorption promoter is not particularly limited as long as it is a compound that has been conventionally recognized to have an absorption promoting action to the skin, and examples thereof include an aliphatic alcohol having 6 to 20 carbon atoms, an aliphatic ether having 6 to 20 carbon atoms, a fatty acid having 6 to 20 carbon atoms, a fatty acid ester having 6 to 20 carbon atoms, a fatty acid amide having 6 to 20 carbon atoms, glycerin, glycerin fatty acid esters, propylene glycols, propylene glycol fatty acid esters, polyethylene glycol and polyethylene glycol fatty acid esters, aromatic organic acid, aromatic alcohol, aromatic organic acid ester, aromatic organic ether (the above compounds may be saturated or unsaturated, may be linear or branched, and may include a cyclic structure), lactic acid esters, acetic acid esters, monoterpene-based compounds, sesquiterpene-based compounds, Azone®, Azone derivatives, sorbitan fatty acid esters (Span®-based), polysorbate-based (Tween®-based), polyoxyethylene hydrogenated castor oils, polyoxyethylene alkyl ethers, sucrose fatty acid esters, and vegetable oils. Specific examples of absorption promoters include caprylic acid, capric acid, caproic acid, lauric acid, myristic acid, palmitic acid, stearic acid, isostearic acid, oleic acid, linoleic acid, linolenic acid, lauryl alcohol, myristyl alcohol, oleyl alcohol, isostearyl alcohol, cetyl alcohol, methyl laurate, hexyl laurate, diethanolamide laurate, isopropyl myristate, myristyl myristate, octyldodecyl myristate, cetyl palmitate, isopropyl palmitate, salicylic acid, methyl salicylate, ethylene glycol salicylate, cinnamic acid, methyl cinnamate, cresol, cetyl lactate, lauryl lactate, ethyl acetate, propyl acetate, geraniol, thymol, eugenol, terpineol, 1-menthol, borneol, d-limonene, isoeugenol, isoborneol, nerol, dl-camphor, glycerin monocaprylate, glycerin monocaprate, glycerin monolaurate, glycerin monooleate, sorbitan monolaurate, sucrose monolaurate, polysorbate 20, propylene glycol, propylene glycol monolaurate, polyethylene glycol monolaurate, polyethylene glycol monostearate, polyoxyethylene lauryl ether, Nikkol® HCO-60 (Nikko Chemicals, Co., Ltd.), Pyrothiodecane®, olive oil, and sorbitan monooleate. The absorption promoters may be used alone or in combination of two or more.

When the adhesive layer contains an absorption promoter, the content of the absorption promoter is preferably 0 to 30 mass %, more preferably 0 to 20 mass % based on the total mass of the adhesive layer.

Examples of the stabilizer include tocopherol and its ester derivatives, ascorbic acid and its ester derivatives, dibutylhydroxytoluene, butylhydroxyanisole, 2-mercaptobenzimidazole and the like. The stabilizers may be used alone or in combination of two or more.

When the adhesive layer contains a stabilizer, the content of the stabilizer is preferably 0 to 5 mass %, and more preferably 0 to 3 mass % based on the total mass of the adhesive layer.

The crosslinking agent is not particularly limited, and preferable examples include amino resins, phenol resins, epoxy resins, alkyd resins, thermosetting resins such as unsaturated polyesters, isocyanate compounds, blocked isocyanate compounds, organic crosslinking agents, inorganic crosslinking agents such as metals and metal compounds.

The preservative is not particularly limited, and preferred examples include ethyl p-hydroxybenzoate, propyl p-hydroxybenzoate, butyl p-hydroxybenzoate and the like.

The filler is not particularly limited, and preferred examples include calcium carbonate, magnesium carbonate, silicates (aluminum silicate, calcium silicate, magnesium silicate, etc.), cellulose derivatives (hydroxypropyl cellulose, hydroxypropylmethyl cellulose, carboxymethyl cellulose, etc.).

The mixing ratio of the rubber-based adhesive and the acrylic-based adhesive of the present invention is 9:1 to 1:9, preferably 9:1 to 3:7, and more preferably 9:1 to 5:5. In addition, the mixing ratio of the alicyclic saturated hydrocarbon resin and the terpene-based resin of the present invention is preferably 10:1 to 1:10, more preferably 5:1 to 1:5, and particularly preferably 2:1 to 1:2.

The patch may further comprise a release liner. The release liner is laminated on the surface of the adhesive layer opposite to the backing. The provision of the release liner tends to reduce attachment of dust or the like to the adhesive layer during storage.

The material of the release liner is not particularly limited, and films generally known to those skilled in the art can be used. Examples of material for the release liner include polyesters such as polyethylene terephthalate and polyethylene naphthalate; polyolefins such as polyethylene and polypropylene; a film of polyvinyl chloride, polyvinylidene chloride, etc.; a laminated film of high-quality paper and polyolefins; a film of Nylon®, aluminum and the like. The material of the release liner is preferably polypropylene or polyethylene terephthalate.

In one embodiment, the adhesive layer of the patch of the invention contains methylphenidate, a rubber-based adhesive and an acrylic-based adhesive, and may further contain a silicic acid compound. By containing the silicic acid compound, phase separation of the rubber-based adhesive and the acrylic-based adhesive can be significantly suppressed, and the physical properties as a preparation and the handleability of the patch can be further improved. Examples of the silicic acid compound include anhydrous silicic acid, calcium silicate, magnesium silicate, aluminum silicate, magnesium aluminum silicate, magnesium aluminate silicate, and sodium magnesium silicate, etc.; and calcium silicate is particularly preferable. As the calcium silicate, for example, a porous one can be used. Specifically, Florite® R (by Tomita Pharmaceutical Co., Ltd., trade name), SIPERANT® 880 (by Evonik Industries AG, trade name), Calcium Silicate (by Spectrum Chemical Co., Ltd., trade name) and the like can be used. The content of the calcium silicate can be appropriately set by those skilled in the art in consideration of sufficient preparation characteristics of the patch, and based on the total amount of the adhesive layer, it is preferably 0.1 to 15 mass %, more preferably 0.5 to 10 mass %, furthermore preferably 1 to 5 mass %, and particularly preferably 2.5 to 5 mass %.

Next, an example of the method for producing the patch of the present invention will be described.

First, a mixture for forming an adhesive layer is prepared. Using a mixer, the above-mentioned methylphenidate, a rubber-based adhesive base, an acrylic-based adhesive base, and other components are dissolved or dispersed in a solvent of the adhesive base to obtain a mixture for forming an adhesive layer.

As a solvent for the adhesive base, toluene, hexane, ethyl acetate, cyclohexane, heptane, butyl acetate, ethanol, methanol, xylene, isopropanol, etc. may be used. These may be appropriately selected depending on the components to be dissolved or dispersed, and one kind may be used alone, or two or more kinds may be mixed and used in combination.

Then, the obtained mixture for forming the adhesive layer is directly spread on a backing to form an adhesive layer, and then a release liner for protecting the adhesive layer is adhered to the adhesive layer; or said mixture is spread on a paper or film that has been subjected to a release treatment to form an adhesive layer, and then placing a backing on it and transfer the adhesive onto the backing by pressure bonding to obtain a patch.

### EXAMPLES

<Cold Flow Test>

[Experimental Method]

Methylphenidate-containing patches shown in Tables 1-1 to 1-8 were produced, then these patches were respectively put into packaging bags and stored at room temperature; after 10 months, cold flow of the preparation from four sides was visually observed. Cold flow scores were set as follows.
1: No cold flow
2: Very slight cold flow
3: Cold flow from less than one side
4: Cold flow from less than two sides
5: Cold flow from two sides or more
6: Cold flow from all sides

[Experimental Result]

The results of the cold flow test are shown in Tables 1-1 to 1-8. For all of the patches (Examples 1 to 35) containing the rubber-based adhesive and the acrylic-based adhesive in the mixing ratio of 9:1 to 1:9, the cold flow score was about 1 to 3, indicating excellent effect of suppressing cold flow. It was also shown that the cold flow score changed depending on which materials were used for the rubber-based adhesive and the acrylic-based adhesive in the patch; in particular, the cold flow score was 1 for those containing SIS in the rubber-based adhesive and alicyclic saturated hydrocarbon resin (ARKON) and/or a terpene-based resin (YS resin) in the tackifying resin (Examples 26 to 30), and for those containing SIS, alicyclic saturated hydrocarbon resin, and terpene-based resin in the rubber-based adhesive (Example 33, etc.), indicating excellent effect of suppressing cold flow.

Here, the degree of cold flow of the preparations after 10 months at room temperature was similar to that of the preparations stored at 60° C. for 1 week or at 40° C. for 1 to 3 months.

TABLE 1-1
Results of cold flow test (Comparative Examples)

| | Com. Ex. | Com. Ex. |
|---|---|---|
| Component | 1 | 2 |
| Methylphenidate (mass %) | 20 | 20 |
| SIS (mass %) | 29 | 0 |
| Alicyclic saturated hydrocarbon | 31 | 0 |
| resin (mass %) | | |
| Terpene-based resin (mass %) | 20 | 0 |
| MAS-811 (mass %) | 0 | 80 |

| | |
|---|---|
| Total (mass %) | 100 |

| | | |
|---|---|---|
| Rubber-based adhesive: | 10:0 | 0:10 |
| Acrylic-based adhesive | | |
| Cold flow score | 4 | 6 |

TABLE 1-2
Results of cold flow test (Examples 1-5)

| | Ex. | Ex. | Ex. | Ex. | Ex. |
|---|---|---|---|---|---|
| Component | 1 | 2 | 3 | 4 | 5 |
| Methylphenidate (mass %) | 20 | 20 | 20 | 20 | 20 |
| SIS (mass %) | 38.4 | 29.9 | 21.3 | 12.8 | 4.27 |
| Alicyclic saturated | 33.6 | 26.1 | 18.7 | 11.2 | 3.73 |
| hydrocarbon resin (mass %) | | | | | |
| MAS-811 (mass %) | 8 | 24 | 40 | 56 | 72 |
| Total (mass %) | 100 | | | | |
| Rubber-based adhesive: | 9:1 | 7:3 | 5:5 | 3:7 | 1:9 |
| Acrylic-based adhesive | | | | | |
| Cold flow score | 1 | 1 | 2 | 3 | 3 |

TABLE 1-3
Results of cold flow test (Examples 6-10)

| | Ex. | Ex. | Ex. | Ex. | Ex. |
|---|---|---|---|---|---|
| Component | 6 | 7 | 8 | 9 | 10 |
| Methylphenidate (mass %) | 20 | 20 | 20 | 20 | 20 |
| SIS (mass %) | 38.4 | 29.9 | 21.3 | 12.8 | 4.27 |
| Terpene-based resin (mass %) | 33.6 | 26.1 | 18.7 | 11.2 | 3.73 |
| MAS-811 (mass %) | 8 | 24 | 40 | 56 | 72 |
| Total (mass %) | 100 | | | | |
| Rubber-based adhesive: | 9:1 | 7:3 | 5:5 | 3:7 | 1:9 |
| Acrylic-based adhesive | | | | | |
| Cold flow score | 1 | 1 | 2 | 2 | 3 |

TABLE 1-4
Results of cold flow test (Examples 11-15)

| | Ex. | Ex. | Ex. | Ex. | Ex. |
|---|---|---|---|---|---|
| Component | 11 | 12 | 13 | 14 | 15 |
| Methylphenidate (mass %) | 20 | 20 | 20 | 20 | 20 |
| SIS (mass %) | 38.4 | 29.9 | 21.3 | 12.8 | 4.27 |
| Alicyclic saturated | 33.6 | 26.1 | 18.7 | 11.2 | 3.73 |
| hydrocarbon resin (mass %) | | | | | |
| Duro-Tak87-4098 (mass %) | 8 | 24 | 40 | 56 | 72 |
| Total (mass %) | 100 | | | | |
| Rubber-based adhesive: | 9:1 | 7:3 | 5:5 | 3:7 | 1:9 |
| Acrylic-based adhesive | | | | | |
| Cold flow score | 1 | 1 | 1 | 2 | 2 |

TABLE 1-5
Results of cold flow test (Examples 16-20)

| | Ex. | Ex. | Ex. | Ex. | Ex. |
|---|---|---|---|---|---|
| Component | 16 | 17 | 18 | 19 | 20 |
| Methylphenidate (mass %) | 20 | 20 | 20 | 20 | 20 |
| SIS (mass %) | 38.4 | 29.9 | 21.3 | 12.8 | 4.27 |
| Alicyclic saturated | 33.6 | 26.1 | 18.7 | 11.2 | 3.73 |
| hydrocarbon resin (mass %) | | | | | |
| Duro-Tak87-4287 (mass %) | 8 | 24 | 40 | 56 | 72 |
| Total (mass %) | 100 | | | | |
| Rubber-based adhesive: | 9:1 | 7:3 | 5:5 | 3:7 | 1:9 |
| Acrylic-based adhesive | | | | | |
| Cold flow score | 1 | 1 | 1 | 2 | 2 |

TABLE 1-6
Results of cold flow test (Examples 21-25)

| | Ex. | Ex. | Ex. | Ex. | Ex. |
|---|---|---|---|---|---|
| Component | 21 | 22 | 23 | 24 | 25 |
| Methylphenidate (mass %) | 20 | 20 | 20 | 20 | 20 |
| SIS (mass %) | 38.4 | 29.9 | 21.3 | 12.8 | 4.27 |
| Terpene-based resin (mass %) | 33.6 | 26.1 | 18.7 | 11.2 | 3.73 |
| Duro-Tak87-4098 (mass %) | 8 | 24 | 40 | 56 | 72 |
| Total (mass %) | 100 | | | | |
| Rubber-based adhesive: | 9:1 | 7:3 | 5:5 | 3:7 | 1:9 |
| Acrylic-based adhesive | | | | | |
| Cold flow score | 1 | 1 | 1 | 2 | 2 |

TABLE 1-7
Results of cold flow test (Examples 26-30)

| | Ex. | Ex. | Ex. | Ex. | Ex. |
|---|---|---|---|---|---|
| Component | 26 | 27 | 28 | 29 | 30 |
| Methylphenidate (mass %) | 20 | 20 | 20 | 20 | 20 |
| SIS (mass %) | 38.4 | 29.9 | 21.3 | 12.8 | 4.27 |
| Terpene-based resin (mass %) | 33.6 | 26.1 | 18.7 | 11.2 | 3.73 |
| Duro-Tak87-4287 (mass %) | 8 | 24 | 40 | 56 | 72 |
| Total (mass %) | 100 | | | | |
| Rubber-based adhesive: | 9:1 | 7:3 | 5:5 | 3:7 | 1:9 |
| Acrylic-based adhesive | | | | | |
| Cold flow score | 1 | 1 | 1 | 1 | 1 |

TABLE 1-8
Results of cold flow test (Examples 31-35)

| | Ex. | Ex. | Ex. | Ex. | Ex. |
|---|---|---|---|---|---|
| Component | 31 | 32 | 33 | 34 | 3 5 |
| Methylphenidate (mass %) | 20 | 20 | 20 | 20 | 20 |
| SIS (mass %) | 38.4 | 21.3 | 4.28 | 21.3 | 21.3 |
| Alicyclic saturated | 16.8 | 9.35 | 1.86 | 12.47 | 6.23 |
| hydrocarbon resin (mass %) | | | | | |
| Terpene-based resin (mass %) | 16.8 | 9.35 | 1.86 | 6.23 | 12.47 |
| MAS-683 (mass %) | 8 | 40 | 72 | 40 | 40 |
| Total (mass %) | 100 | | | | |
| Rubber-based adhesive: | 9:1 | 3:7 | 1:9 | 5:5 | 5:5 |
| Acrylic-based adhesive | | | | | |
| Alicyclic saturated | 1:1 | 1:1 | 1:1 | 2:1 | 1:2 |
| hydrocarbon resin: | | | | | |
| Terpene-based resin | | | | | |
| Cold flow score | 1 | 2 | 1 | 2 | 1 |

<Phase Separation Test 1>

[Experimental Method]

The phase separation of the preparations was visually observed at room temperature during or immediately after production of the patches of Examples 1 to 5 and 11 to 20 described above. The evaluation criteria were set as follows. For convenience, those with a rating of 80 or more were judged to have excellent production suitability.
100: No phase separation in appearance (uniform preparation)
80: Slight pattern of phase separation is observed
60: Pattern of phase separation is observed
40: Phase separation is remarkable
20: Separation at the time of application
0: Application impossible

[Experimental Result]

The results of the phase separation test are shown in Tables 2-1 to 2-3. The phase separation scores of all the patches of Examples 1 to 5, 11, 14, 16 to 20 were 80 or more.

While the preparations with an evaluation of 20 or more and less than 80 showed phase separation in appearance, permeability of the drug and adhesiveness of the preparations were about the same as the preparations without phase separation.

TABLE 2-1
Results of phase separation test (Examples 1-5)

| | Ex. | Ex. | Ex. | Ex. | Ex. |
|---|---|---|---|---|---|
| Component | 1 | 2 | 3 | 4 | 5 |
| Methylphenidate (mass %) | 20 | 20 | 20 | 20 | 20 |
| SIS (mass %) | 38.4 | 29.9 | 21.3 | 12.8 | 4.27 |
| Alicyclic saturated | 33.6 | 26.1 | 18.7 | 11.2 | 3.73 |
| hydrocarbon resin (mass %) | | | | | |
| MAS-811 (mass %) | 8 | 24 | 40 | 56 | 72 |
| Total (mass %) | 100 | | | | |
| Rubber-based adhesive: | 9:1 | 7:3 | 5:5 | 3:7 | 1:9 |
| Acrylic-based adhesive | | | | | |
| Phase separation score | 90 | 85 | 85 | 85 | 90 |

TABLE 2-2
Results of phase separation test (Examples 11-15)

| | Ex. | Ex. | Ex. | Ex. | Ex. |
|---|---|---|---|---|---|
| Component | 11 | 12 | 13 | 14 | 15 |
| Methylphenidate (mass %) | 20 | 20 | 20 | 20 | 20 |
| SIS (mass %) | 38.4 | 29.9 | 21.3 | 12.8 | 4.27 |
| Alicyclic saturated | 33.6 | 26.1 | 18.7 | 11.2 | 3.73 |
| hydrocarbon resin (mass %) | | | | | |
| Duro-Tak87-4098 (mass %) | 8 | 24 | 40 | 56 | 72 |
| Total (mass %) | 100 | | | | |
| Rubber-based adhesive: | 9:1 | 7:3 | 5:5 | 3:7 | 1:9 |
| Acrylic-based adhesive | | | | | |
| Phase separation score | 85 | 30 | 60 | 80 | 20 |

TABLE 2-3
Results of phase separation test (Examples 16-20)

| | Ex. | Ex. | Ex. | Ex. | Ex. |
|---|---|---|---|---|---|
| Component | 16 | 17 | 18 | 19 | 20 |
| Methylphenidate (mass %) | 20 | 20 | 20 | 20 | 20 |
| SIS (mass %) | 38.4 | 29.9 | 21.3 | 12.8 | 4.27 |
| Alicyclic saturated | 33.6 | 26.1 | 18.7 | 11.2 | 3.73 |
| hydrocarbon resin (mass %) | | | | | |
| Duro-Tak87-4287 (mass %) | 8 | 24 | 40 | 56 | 72 |
| Total (mass %) | 100 | | | | |
| Rubber-based adhesive: | 9:1 | 7:3 | 5:5 | 3:7 | 1:9 |
| Acrylic-based adhesive | | | | | |
| Phase separation score | 90 | 85 | 80 | 85 | 90 |

<Phase Separation Test 2>

[Experimental Method]

The patches containing methylphenidate with the compositions shown in Tables 3-1 to 3-4 were produced as those wherein the rubber-based adhesive SIS (100 parts by weight) and the alicyclic saturated hydrocarbon resin (Alcon) (87.5 parts by weight) were mixed, then phase separation of these patches was evaluated in the same manner as in the phase separation test 1 above.

[Experimental Result]

The results of the phase separation test are shown in Tables 3-1 to 3-4. In contrast to the results of the above-mentioned phase separation test 1, all of the patches containing calcium silicate (Examples 36, 42-47) had a phase separation score of 100, indicating excellent effect of suppressing phase separation. Meanwhile, all of the patches without calcium silicate and containing methylphenidate and a rubber-based adhesive or an acrylic-based adhesive (Reference Examples 1 to 5) had a phase separation score of 100, so the phase separation was not recognized.

TABLE 3-1
Results of phase separation test (Reference Examples 1-5)

| | Ref. Ex. | Ref. Ex. | Ref. Ex. | Ref. Ex. | Ref. Ex. |
|---|---|---|---|---|---|
| Component | 1 | 2 | 3 | 4 | 5 |
| Methylphenidate(mass %) | 20 | 20 | 20 | 20 | 20 |
| Calcium silicate(mass %) | 0 | 0 | 0 | 0 | 0 |
| Rubber-based | 80 | 0 | 0 | 0 | 0 |
| adhesive (mass %) | | | | | |
| MAS-683 (mass %) | 0 | 80 | 0 | 0 | 0 |
| MAS-811 (mass %) | 0 | 0 | 80 | 0 | 0 |
| Duro-Tak87-4098(mass %) | 0 | 0 | 0 | 80 | 0 |
| Duro-Tak87-4287(mass %) | 0 | 0 | 0 | 0 | 80 |
| Total (mass %) | 100 | | | | |
| Rubber-based adhesive: | 10:0 | 0:10 | 0:10 | 0:10 | 0 :10 |
| Acrylic-based adhesive | | | | | |
| Phase separation score | 100 | 100 | 100 | 100 | 100 |

TABLE 3-2
Results of phase separation test (Examples 36-41)

| | Ex. | Ex. | Ex. | Ex. | Ex. | Ex. |
|---|---|---|---|---|---|---|
| Component | 36 | 37 | 38 | 39 | 40 | 41 |
| Methylphenidate (mass %) | 20 | 20 | 20 | 20 | 20 | 20 |
| Calcium silicate (mass %) | 1 | 0 | 0 | 0 | 0 | 0 |
| Rubber-based adhesive | 39.5 | 72 | 56 | 40 | 24 | 8 |
| (mass %) | | | | | | |
| MAS-683 (mass %) | 39.5 | 8 | 24 | 40 | 56 | 72 |
| Total (mass %) | 100 | | | | | |
| Rubber-based adhesive: | 5:5 | 9:1 | 7:3 | 5:5 | 3:7 | 1:9 |
| Acrylic-based adhesive | | | | | | |
| Phase separation score | 100 | 90 | 50 | 30 | 85 | 90 |

TABLE 3-3
Results of phase separation test (Examples 42-45)

| | Ex. | Ex. | Ex. | Ex. |
|---|---|---|---|---|
| Component | 42 | 43 | 44 | 45 |
| Methylphenidate (mass %) | 20 | 20 | 20 | 20 |
| Calcium silicate (mass %) | 0.1 | 1 | 2.5 | 5 |
| Rubber-based adhesive (mass %) | 39.95 | 39.5 | 38.75 | 37.5 |
| MAS-811 (mass %) | 39.95 | 39.5 | 38.75 | 37.5 |
| Total (mass %) | 100 | | | |
| Rubber-based adhesive: | 5:5 | 5:5 | 5:5 | 5:5 |
| Acrylic-based adhesive | | | | |
| Phase separation score | 100 | 100 | 100 | 100 |

TABLE 3-4
Results of phase separation test (Examples 46, 47)

| | Ex. | Ex. |
|---|---|---|
| Component | 46 | 47 |
| Methylphenidate (mass %) | 20 | 20 |
| Calcium silicate (mass %) | 1 | 1 |
| Rubber-based adhesive (mass %) | 39.5 | 39.5 |
| Duro-Tak87-4098 (mass %) | 39.5 | 0 |
| Duro-Tak87-4287 (mass %) | 0 | 39.5 |

| | |
|---|---|
| Total (mass %) | 100 |

| | | |
|---|---|---|
| Rubber-based adhesive: | 5:5 | 5:5 |
| Acrylic-based adhesive | | |
| Phase separation score | 100 | 100 |

<Phase Separation Test 3>

[Experimental Method]

Patch containing methylphenidate with the compositions shown in Table 4 were produced, then the phase separation of these patches was evaluated in the same manner as in the phase separation test 1 above.

[Experimental Result]

Table 4 shows the results of the phase separation test. Examples 48 and 49 correspond to those that contain calcium silicate in Examples 12 and 15, respectively. In both of Examples 48 and 49 containing calcium silicate, the phase separation score was 100, indicating an excellent effect of suppressing phase separation.

TABLE 4
Results of phase separation test (Examples 48, 49)

| | Ex. | Ex. |
|---|---|---|
| Component | 48 | 49 |
| Methylphenidate (mass %) | 20 | 20 |
| Calcium silicate (mass %) | 1 | 1 |
| SIS (mass %) | 29.5 | 4.22 |
| Alicyclic hydrocarbon | 29.5 | 3.68 |
| resin (mass %) | | |
| Duro-Tak87-4098 (mass %) | 23.7 | 71.1 |

| | |
|---|---|
| Total (mass %) | 100 |

| | | |
|---|---|---|
| Rubber-based adhesive: | 7:3 | 1:9 |
| Acrylic-based adhesive | | |
| Phase separation score | 100 | 100 |

1. A patch comprising a backing layer and an adhesive layer, wherein
the adhesive layer contains methylphenidate, a rubber-based adhesive and an acrylic-based adhesive, and
the mixing ratio of the rubber-based adhesive and the acrylic-based adhesive is 9:1 to 1:9.

2. The patch according to claim 1, wherein the rubber-based adhesive contains a styrene-isoprene-styrene block copolymer.

3. The patch according to claim 1, wherein the rubber-based adhesive contains a tackifying resin.

4. The patch according to claim 1, wherein the acrylic-based adhesive does not have a carboxyl group.

5. The patch according to claim 1, wherein the rubber-based adhesive contains an alicyclic hydrocarbon resin and/or a terpene-based resin as a tackifying resin.

6. The patch according to claim 1, wherein the adhesive layer contains methylphenidate in a proportion of 10 to 30 mass % relative to the total amount of the adhesive layer.

7. The patch according to claim 1, wherein the mixing ratio of the rubber-based adhesive and the acrylic-based adhesive is 9:1 to 3:7.

8. The patch according to claim 5, wherein the mixing ratio of the alicyclic hydrocarbon resin and the terpene-based resin is 2:1 to 1:2.
